(19) United States
(12) Patent Application Publication
(10) Pub. No.: US 2009/0068730 A1
(43) Pub. Date: Mar. 12, 2009

(54) FRICTION FIT CONTACT PLATE

(76) Inventor: Victor Dayktn, Niagara On The Lake (CA)

Correspondence Address: DENNISON ASSOCIATES, 133 RICHMOND STREET WEST, SUITE 301, TORONTO, ON, M5H 2L7, CA

(21) Appl. No.: 11/853,014

(22) Filed: Sep. 10, 2007

(51) Int. Cl. C12M 1/22

(52) U.S. Cl. 435/307.1

(57) ABSTRACT

A contact plate for growing cell cultures, bacteria cultures, and the like, comprising a base and a cover. The base has a bottom wall for holding the culture medium and the like, and a circumferential sidewall extending upwardly from the bottom wall and attached thereto. The base further incorporates a flange extending outwardly from the lower edge of the outer periphery of the sidewall. The outer periphery of the base sidewall is provided with a generally perpendicular surface along the lower edge thereof The cover has a top wall, which has a larger diameter than the base sidewall, and a circumferential sidewall extending downwardly from the top wall and attached thereto. The cover is provided with a plurality of projections on the inner surface of the sidewall to engage the generally perpendicular surface of the lower edge of the outer periphery of the base sidewall. The projections have a diameter less than the diameter of the generally perpendicular surface of the lower edge of the outer periphery of the base sidewall to provide a biasing force to retain the cover on the base when the projections engage the generally perpendicular surface of the lower edge of the outer periphery of the base sidewall.

### FIELD OF THE INVENTION

The present invention relates to contact plates, and more particularly to friction fit contact plates which have a means for releasably holding the cover to the base by a friction fitting.

### BACKGROUND OF THE INVENTION

Contact plates, also known as culture dishes or Petri dishes, are widely known and used in laboratories for growing cell cultures, bacteria cultures, and the like. Conventional contact plates are comprised of two parts: 1 a base, which is typically a shallow open-ended cylindrical container; and 2 a cover, which is wider in diameter than the base, and fits over and covers the base.

Conventional contact plates are difficult for the user to lift and move because the cover is wider in diameter than the base and is loosely positioned over the base. In particular, the user must ensure that he or she has a firm grasp on both the base and the cover when lifting and moving the contact plate. Accordingly, either both hands are required to grasp and lift the contact plate, or the user must slide the entire contact plate off the edge of the working surface to grasp the contact plate. Alternatively, in order to lift and support the base, the user must tilt the contact plate to facilitate the positioning of his or her fingers underneath the base. This is undesirable because the tilting can shift and disturb the contents of the contact plate. Tilting of the contact plate may also cause the cover to slide; thus, making it awkward for the user to get a firm grasp on the contact plate.

Conventional contact plates are prone to accidental or inadvertent opening which may contaminate the culture. Conventional contact plates are often stacked together on the working surface, in the incubator, or in the storage area for space efficiency. As the cover of a conventional contact plate is loosely positioned over the base, the cover can be accidentally opened when the stacked contact plates are handled. In addition, the cover of a contact plate may open if the contact plate is jostled during handling. Thus, it is highly desirable to have contact plates which prevent accidental openings.

Various contact plates have been provided in which the cover is placed over the base in a snug or tight fit. For example, U.S. Pat. No. 5,725,123 discloses a cover with individual elevations on the inner surface of the cover apron, which press against the outer surface of the base wall to secure the cover to the container. Similarly, U.S. Pat. No. 6,602,704 discloses a contact plate with a cover having a plurality of ribs on the inner surface which press against the outer surface of the base wall to secure the cover to the container. However, it has been found that these prior art arrangements have elevations or ribs which ride on an upwardly sloping surface. Once media lubricates the surface, the media acts as a lubricant and friction fit engagement is reduced and almost eliminated, leading to possible accidental opening of the contact plate.

There thus remains a need for a friction fit contact plate which overcomes the disadvantages of the prior art.

### SUMMARY OF THE INVENTION

The present invention is directed to a contact plate comprising a base and a cover. The base has a bottom wall for holding the culture medium and the like, and a circumferential sidewall extending upwardly from the bottom wall and attached thereto. The base further incorporates a flange extending outwardly from the lower edge of the outer periphery of the sidewall. The outer periphery of the base sidewall is provided with a generally perpendicular surface along the lower edge thereof. The cover has a top wall, which has a larger diameter than the base sidewall, and a circumferential sidewall extending downwardly from the top wall and attached thereto. The cover is provided with a plurality of projections on the inner surface to engage the generally perpendicular surface of the lower edge of the outer periphery of the base sidewall. The projections have a diameter less than the diameter of the generally perpendicular surface of the lower edge of the outer periphery of the base sidewall to provide a biasing force to retain the cover on the base when the projections engage the generally perpendicular surface of the lower edge of the outer periphery of the base sidewall.

In an aspect of the inventions the projections are angled slightly inwardly at the lower edge to provide a contact surface for engagement with the generally perpendicular surface of the lower edge of the outer periphery of the base sidewall.

### BRIEF DESCRIPTION OF THE DRAWINGS

Preferred embodiments of the invention are shown in the drawings, wherein:

FIG. 1 is an exploded perspective view of the contact plate of the present invention showing the cover and base;.

FIG. 2 is a cross-sectional side view of the contact plate of FIG. 1 with the cover separated from the base;

FIG. 3 is a cross-sectional side view of the contact plate of FIG. 1 with the cover in the process of being placed on the base;

FIG. 4 is a cross-sectional side view of the contact plate of FIG. 1 with the cover and base secured together; and

FIG. 5 shows a detail view of the engagement means of the contact plate of FIG. 1.

### DETAILED DESCRIPTION OF THE PREFERRED EMBODIMENTS

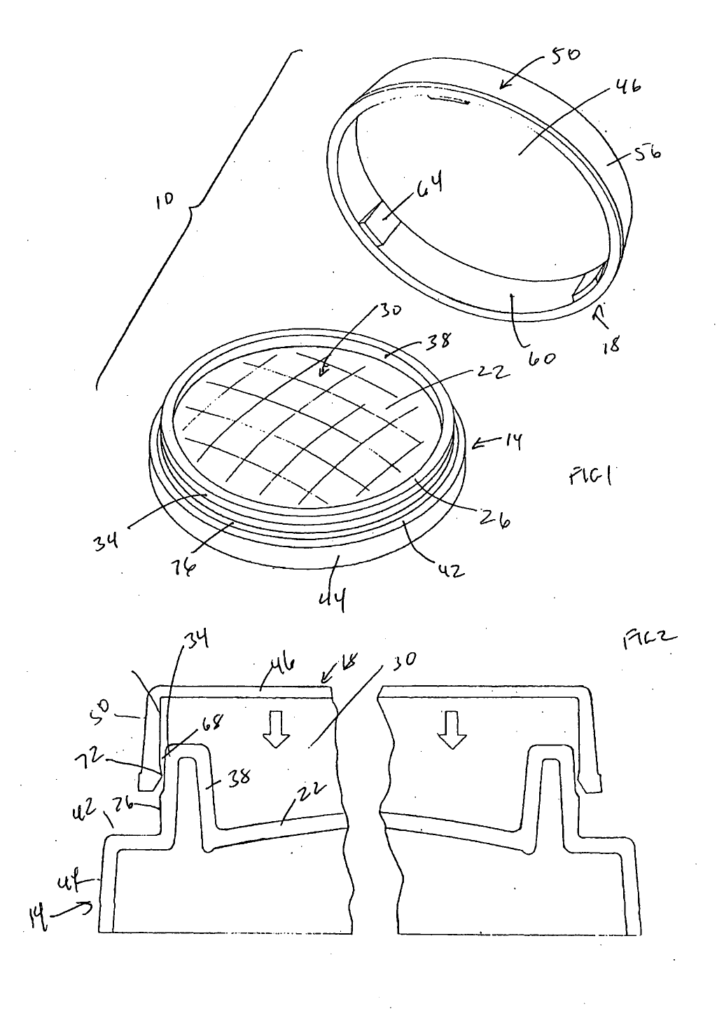

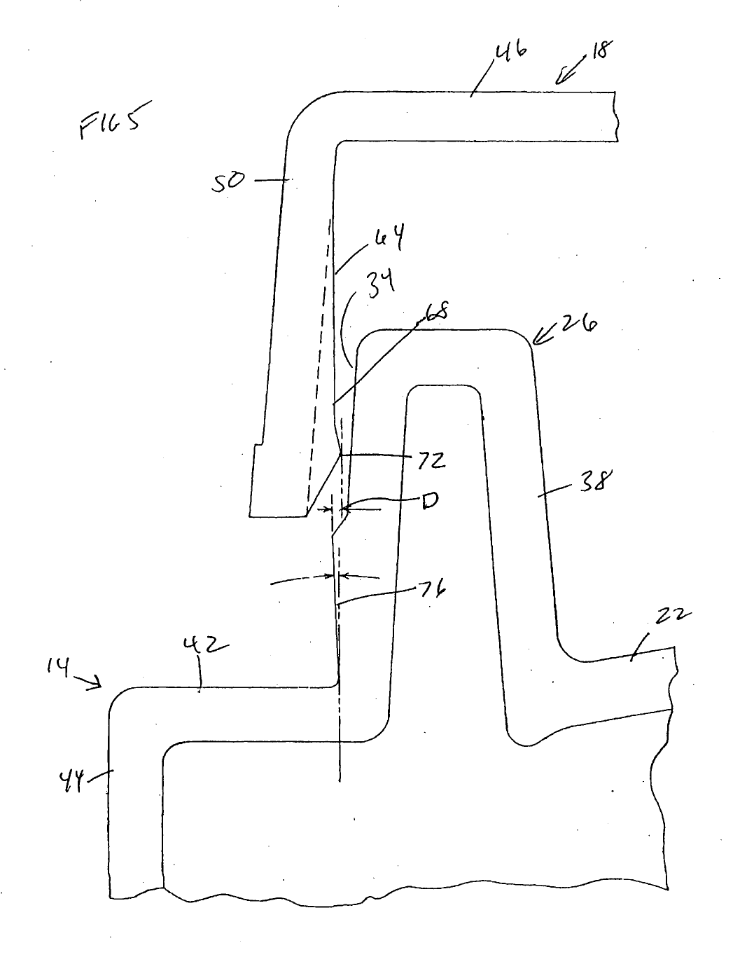

With reference to FIGS. 1 to 5, a contact plate is generally indicated with the numeral 10. The contact plate 10 of the present invention includes a base 14 and a cover 18. The contact plate 10 is preferably fabricated from a plastic material. However, it can be appreciated that other suitable materials can be used. At least the cover 18, but preferably both the cover 18 and the base 14, is fabricated of a transparent material to enable the user to view the contents of the contact plate. It can also be appreciated that the size of the contact plate is not limited to any specific dimensions.

The base 14 includes a bottom wall 22 and a side wall 26, which extends upward from the bottom wall 22 and extends continuously along its entire periphery, to form an upward facing receptacle 30 for holding the culture medium and the like. Preferably, the base 14 is formed from an unitary integrally molded piece of plastic wherein the bottom wall 22 is integrally formed with the side wall 26. The side wall 26 is comprised of a outer surface area 34 and an inner surface area 38. The bottom wall 22 may be provided with suitable lines or a grid, as shown in the figures, so as to facilitate the counting of the culture in the conventional manner.

The base 14 provides at least one flange 42 extending outwardly from the outer surface area 34 of the side wall 26 and attached thereto. The flange 42 is preferably integrally formed with the side wall 26 and has a flange surface 43. In a preferred embodiment of the invention, as shown in the figures, the flange 42 extends outwardly from the outer surface area 34 of the side wall 26 and extends continuously along the entire periphery. Surrounding the contact plate 10 and extending downwardly from the perimeter of the flange 42 is a short skirt 44, which may serve as a surface for gripping and manipulating the contact plate.

The cover 18 has a top wall 46 and a side wall 50, which extends downwardly from the top wall 46 and extends continuously along its entire periphery. Preferably, the cover 18 is formed from a unitary integrally molded piece of plastic wherein the top wall 46 is integrally formed with the side wall 50. The side wall 50 has an outer surface area 56 and an inner surface area 60.

As best shown in the Figures, the cover 18 is positioned over the base receptacle 30 in the closed position. The top wall 46 has a larger diameter than the base side wall 26. The cover side wall 50 is of a size and configuration that it surrounds and overlaps the base side wall 26, wherein the edge 62 of the cover side wall 50 rests on and is supported by the flange surface 43. The cover side wall 50 preferably has a height greater than the height of the base side wall 26.

As shown in the Figures, the cover 18 and base 14 have a means for releasably holding the cover 18 to the base 14 of the contact plate 10 when the cover 18 is in the closed position, wherein the cover 18 is held to the base 14 by a positive displacement friction fit.

In the embodiment shown in the figures, the cover 18 includes a plurality of inwardly extending projections 64, which extend inwardly from the inner surface area 60. The projections 64 are preferably disposed equaly speced around the inner surface area 60. Preferably, there are at least two projections 64 provided on the inner surface, more prefarably at least three projections 64 and most preferably up to six projections 64. Each projection 64 has a generally perpendicular lower surface 68 with an inwardly angled contact surface 72 adjacent the lower end thereof.

The outer surface 34 of the sidewall 26 of the base 14 is provided with a generally perpendicular surface 76 along the lower edge of the outer surface of the sidewall 34 to provide an engagement surface for the perpendicular lower surface 68 and inwardly angled contact surface 72 of the cover 18.

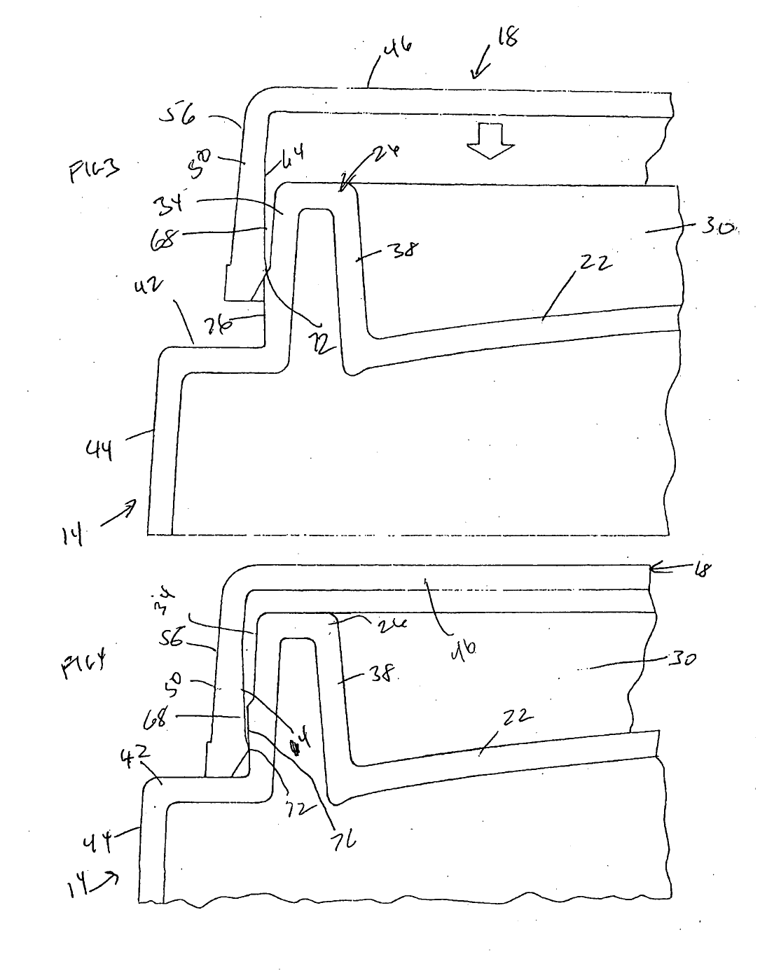

The operation of the friction fit engagement of the contact plate 10 of the present invention will now be explained with reference to FIGS. 2 to 5. As shown in FIG. 5, the projections 64 have a diameter less than the diameter of the generally perpendicular surface 76 of the lower edge of the outer periphery 34 of the base sidewall 26 to provide a biasing force to retain the cover 18 on the base 14 when the projections 64 engage the generally perpendicular surface 76 of the lower edge of the outer periphery 34 of the base sidewall 26. The difference in diameters is represented in FIG. 5 by the letter D. As the cover 18 is placed downwardly over the base 14, the generally perpendicular lower surface 68 and preferably the inwardly angled contact surface 72 of the projections 64 encounter the generally perpendicular lower surface 76 of the outer periphery of the sidewall 26 of the base 14. Since the diameter of the generally perpendicular lower surface 68 and preferably the inwardly angled contact surface 72 of the projections 64 is less than the diameter of the generally perpendicular lower surface 76 of the periphery of the sidewall 26, the projections 64 and hence the sidewall 50 of the cover 18 is biased outwardly as the lower surface of the projection 68 and preferably the inwardly angled contact surface 72 encounters and rides over the generally perpendicular surface 76 of the lower periphery of the sidewall 26 of the base 14. The biasing outwardly of the projections 64 and the cover 18 provides a positive biasing force against the base 14 to releasably hold the cover 18 on the base 14. In order to remove the cover 18, a force greater than this positive biasing force must be exerted to lift the cover 18 off the base 14.

As shown in FIGS. 2 to 5, the provision of the inwardly angled contact surface 72 at the lower edge of the generally perpendicular lower surface 68 of the projections 64, provides an increased biasing force as well as a point contact between the cover 18 and the base 14. This increased biasing and point contact increases the positive biasing force releasably holding the cover I 8 to the base 14 and reduces the likelihood of accidental opening of the contact plate 10.

The contact plate of the present invention provides an easy to use friction fit contact plate which can be easily handled in all of the operation steps commonly employed. The extent of the positive biasing force provide by the structures of the contact plate of the present invention allows the plate to be handled with reduced risk of accidental opening as compared to prior art friction fit contact plates. The contact plate of the present invention can be opened for specimen handling by exerting a force to overcome the positive biasing force. This can easily be accomplished by the technician or automated handling apparatus.

While the contact plate of the present invention provides a positive biasing force greater than the prior art friction fit contact plates, the contact plate can still be used in automatic filling and handling equipment commonly utilized as such equipment can exert sufficient opening force to overcome the positive biasing.

In some applications, it may be desirable to stack a series of contact plates 10 on top of each other. The skirt 44, which surrounds the contact plate 10 and extending downwardly from the perimeter of the flange 42, has a larger diameter than the top wall 46 of the cover 18, thereby providing a means for preventing the shifting of the lower-positioned contact plate. The base 14 may also include a groove which has the same diameter as the top wall 46 of the cover 18, thereby providing a means for restricting the shifting of the lower-positioned contact plate.

Although various preferred embodiments of the present invention have been described herein in detail, it will be appreciated by those skilled in the art, that variations and modifications may be made thereto without departing from the scope of the appended claims.

What is claimed is:

1. A contact plate comprising a base and a cover, the base having a bottom wall for holding a culture medium and the like, and a circumferential sidewall extending upwardly from the bottom wall and attached thereto, the base further incorporates a flange extending outwardly from the lower edge of the outer periphery of the sidewall, the outer periphery of the base sidewall is provided with a generally perpendicular surface along the lower edge thereof, the cover has a top wall, which has a larger diameter than the base sidewall, and a circumferential sidewall extending downwardly from the top wall and attached thereto, the cover is provided with a plurality of projections on the inner surface of the sidewall to engage the generally perpendicular surface of the lower edge of the outer periphery of the base sidewall, the projections having a diameter less than the diameter of the generally perpendicular surface of the lower edge of the outer periphery of the base sidewall to provide a biasing force to retain the cover on the base when the projections engage the generally perpendicular surface of the lower edge of the outer periphery of the base sidewall.

2. A contact plate according to claim 1 wherein the projection is angled slightly inwardly at the lower edge to provide a contact surface for engagement with the generally perpendicular surface of the lower edge of the outer periphery of the base sidewall.
